(19) United States
(12) Patent Application Publication
(10) Pub. No.: US 2025/0367139 A1
(43) Pub. Date: Dec. 4, 2025

(54) COMPOUND HYDROCORTISONE AND NEOMYCIN SULFATE GRANULE AND PREPARATION METHOD THEREOF

(71) Applicant: HEBEI CHANGTIAN PHARMACEUTICAL CO., LTD., Hebei (CN)

(72) Inventors: Xiaorui JIA, Hebei (CN); Meng WU, Hebei (CN); Zhihua YU, Hebei (CN); Dansai WANG, Hebei (CN)

(21) Appl. No.: 18/874,907

(22) PCT Filed: Jun. 15, 2023
(86) PCT No.: PCT/CN2023/100482

§ 371 Date: Dec. 13, 2024

(30) Foreign Application Priority Data
Jun. 15, 2022 (CN) 202210678843.1

(51) Int. Cl. A61K 31/137; A61K 9/16; A61K 31/167; A61K 31/4402; A61K 31/566; A61K 35/413; A61K 47/46; A61K 47/69

(52) U.S. Cl. CPC A61K 31/137; A61K 9/1664; A61K 31/167; A61K 31/4402; A61K 31/566; A61K 35/413; A61K 47/46; A61K 47/6951

(57) ABSTRACT

Provided is a compound hydrocortisone and neomycin sulfate granule, including the following components in parts by mass: 140 parts of acetaminophen, 1 part of chlorpheniramine maleate, 1.2 parts of methylephedrine hydrochloride, 2 to 2.5 parts of an inclusion stabilizer, 3.0 parts of artificial calculus bovis, and 0.5 to 1 parts of a caramel. In a method for preparing the compound hydrocortisone and neomycin sulfate granule, natural Chinese medicinal herbs are subjected to extraction to obtain natural stabilizers for stabilizing low-concentration acetaminophen and chlorpheniramine maleate, and the natural stabilizers are encapsulated with hydroxypropyl-β-cyclodextrin with a specific degree of substitution (DS).

The present application claims priority to the Chinese Patent Application No. CN 202210678843.1 filed with the China National Intellectual Property Administration (CNIPA) on Jun. 15, 2022 and entitled “COMPOUND HYDROCORTISONE AND NEOMYCIN SULFATE GRANULE AND PREPARATION METHOD THEREOF”, which is incorporated herein by reference in its entirety.

### TECHNICAL FIELD

The present disclosure relates to the technical field of pharmaceutical preparations, and in particular to a compound hydrocortisone and neomycin sulfate granule and a preparation method thereof.

### BACKGROUND

Traditional compound hydrocortisone and neomycin sulfate preparations mainly include acetaminophen and chlorpheniramine maleate as functional ingredients, and also include adjuvant components such as artificial calculus bovis. Acetaminophen (also known as paracetamol) is a metabolite of phenacetin in vivo. Acetaminophen is an acetanilide antipyretic analgesic. Acetaminophen could reduce the synthesis and release of prostaglandin E1 (PGE1) by inhibiting the prostaglandin synthase of the hypothalamic thermoregulatory center to cause the peripheral vasodilation and sweating, thereby playing an antipyretic role. Acetaminophen exhibits a similar antipyretic effect to aspirin, but has no obvious anti-inflammatory effect. Acetaminophen could raise a pain threshold by inhibiting the synthesis and release of PGE1, bradykinin, histamine, etc, thereby realizing an analgesic effect. Acetaminophen is a peripheral analgesic drug. When acetaminophen is administered at an excess dose, including a toxic dose, symptoms such as nausea, vomiting, stomach pain, diarrhea, anorexia, and hidrosis might quickly occur.

Chlorpheniramine maleate (also known as chlorpheniramine) is an antihistamine. Chlorpheniramine maleate could exert an anti-allergic action through the antagonism to the H1 receptor. Chlorpheniramine maleate is mainly used for treating rhinitis and skin and mucosal allergies and relieving cold symptoms such as tearing, sneezing, and running nose. In addition to the anti-allergic effect through the antagonism of H1 receptors based on the strong competitive blocking of the histamine H1 receptor on target cells of an allergic reaction, chlorpheniramine maleate could have an anti-M choline receptor effect. Thus, after chlorpheniramine maleate being orally administrated, symptoms such as dry mouth, constipation, sputum thickening, and nasal mucosa dryness might occur. Moreover, chlorpheniramine maleate also has a specified central inhibition effect, such that the adverse reaction of drowsiness might occur after the administration of chlorpheniramine maleate. Therefore, in order to improve the above symptoms caused by chlorpheniramine maleate, an ephedrine stimulant is usually added to a formula. However, because ephedrine is a α and β receptor stimulant, high levels of ephedrine will cause many side effects to the heart and central nervous system. However, if chlorpheniramine maleate and ephedrine are added at low contents in a drug formula, a valid state requiring a stable quality is unsatisfactory. How to ensure the effectiveness of a compound hydrocortisone and neomycin sulfate preparation with low concentrations of chlorpheniramine maleate and ephedrine to avoid the side effects and adverse reactions caused by high concentrations of chlorpheniramine maleate and ephedrine is currently an urgent technical problem to be solved.

Moreover, the artificial calculus bovis added to the compound hydrocortisone and neomycin sulfate preparation is usually produced by mixing an ox bile powder, taurine, bile acid, hyodeoxycholic acid, bilirubin, cholesterol, and trace components. The bilirubin is a bile pigment extracted from the bile. The bilirubin has poor stability in an environment, which greatly affects the stability and medicinal properties of the artificial calculus bovis. How to reduce the loss of bilirubin as a main pharmacodynamic component in the artificial calculus bovis to guarantee the efficacy of the artificial calculus bovis is also a technical problem to be solved in the art.

### SUMMARY

In view of this, the present disclosure is to provide a compound hydrocortisone and neomycin sulfate granule and a preparation method thereof, so as to solve the problems in the prior art. The present disclosure makes it possible to reduce the loss of bilirubin as an active ingredient in artificial calculus bovis while allowing the quality stability of low-concentration chlorpheniramine maleate and ephedrine.

To achieve the above objects of the present disclosure, the present disclosure provides the following technical solutions:

The present disclosure provides a compound hydrocortisone and neomycin sulfate granule, including the following components in parts by mass:

1140 parts of acetaminophen, 1 part of chlorpheniramine maleate, 1.2 parts of methylephedrine hydrochloride, 2 to 2.5 parts of an inclusion stabilizer, 3.0 parts of artificial calculus bovis, and 0.5 to 1 parts of a caramel,
where the inclusion stabilizer is produced by encapsulating a white peony root extract and a licorice extract with hydroxypropyl-β-cyclodextrin.

In some embodiments, the compound hydrocortisone and neomycin sulfate granule include the following components in parts by mass:
140 parts of the acetaminophen, 1 part of the chlorpheniramine maleate, 1.2 parts of the methylephedrine hydrochloride, 2 parts of the inclusion stabilizer, 3.0 parts of the artificial calculus bovis, and 0.5 parts of the caramel.

In some embodiments, the compound hydrocortisone and neomycin sulfate granule include the following components in parts by mass:
140 parts of the acetaminophen, 1 part of the chlorpheniramine maleate, 1.2 parts of the methylephedrine hydrochloride, 2.5 parts of the inclusion stabilizer, 3.0 parts of the artificial calculus bovis, and 1 part of the caramel.

In some embodiments, the compound hydrocortisone and neomycin sulfate granule include the following components in parts by mass:
140 parts of the acetaminophen, 1 part of the chlorpheniramine maleate, 1.2 parts of the methylephedrine hydrochloride, 2.5 parts of the inclusion stabilizer, 3.0 parts of the artificial calculus bovis, and 0.6 parts of the caramel 1.

In some embodiments, the compound hydrocortisone and neomycin sulfate granule include the following components in parts by mass:
t140 parts of the acetaminophen, 1 part of the chlorpheniramine maleate, 1.2 parts of the methylephedrine hydrochloride, 2.5 parts of the inclusion stabilizer, 3.0 parts of the artificial calculus bovis, and 0.7 parts of the caramel.

In some embodiments, the compound hydrocortisone and neomycin sulfate granule include the following components in parts by mass:
140 parts of the acetaminophen, 1 part of the chlorpheniramine maleate, 1.2 parts of the methylephedrine hydrochloride, 2.5 parts of the inclusion stabilizer, 3.0 parts of the artificial calculus bovis, and 0.8 parts of the caramel.

In some embodiments, the white peony root extract is extracted by a process comprising the following steps:
(1) crushing a white peony root to obtain a crushed material, and mixing the crushed material with water to obtain a mixed system; applying a pressure of 120 MPa to 125 MPa continuously for 1 min to 1.5 min and then a pressure of 100 MPa to 105 MPa continuously for 0.5 min to 1 min to the mixed system; and subjecting a resulting system to pressure relief, and then filtration to obtain a filtrate, and collecting the filtrate; and
(2) adjusting an alcohol content of the filtrate to 40% to 42%, standing to produce a precipitate, and discarding the precipitate to obtain a supernatant; further adjusting an alcohol content of the supernatant to 55% to 57%, and subjecting a resulting system to standing and then vacuum filtration to obtain a filter cake; and collecting and drying the filter cake to obtain the white peony root extract.

In some embodiments, a ratio of a mass of the white peony root to a volume of the water is 1 g: 50 mL.

In some embodiments, the licorice extract is extracted by a method including the following steps:
crushing licorice, and adding water as an extraction solvent to a crushed licorice to obtain a mixture; introducing carbon dioxide into the mixture at a temperature of 50° C. to 60° C. and a pressure of 10 MPa to 40 MPa for 20 min to 40 min, and conducting pressure relief to obtain a reaction system; and concentrating and drying the reaction system to obtain the licorice extract.

In some embodiments, a volume ratio of a mixture of the licorice and the water to the carbon dioxide is in a range of 1:1 to 1:5.

In some embodiments, the inclusion stabilizer is prepared by a process comprising the following steps:
(1) dissolving the hydroxypropyl-β-cyclodextrin in an acid solution with a pH of 2.0 to prepare a solution of 4.5 wt % to 5.0 wt %;
(2) dissolving the white peony root extract and the licorice extract in anhydrous ethanol to obtain a mixed extract solution, adding the mixed extract solution dropwise to the solution obtained in step (1), and subjecting a resulting mixture to stirring in a nitrogen atmosphere and inclusion for 7 h to 8 h to obtain a reaction system; and
(3) centrifuging the reaction system obtained in step (2) to obtain a supernatant, and collecting and lyophilizing the supernatant to obtain the inclusion stabilizer.

In some embodiments, a mass ratio of the white peony root extract to the licorice extract is 1:1.5.

In some embodiments, a degree of substitution (DS) of the hydroxypropyl-β-cyclodextrin is 4.2.

The present disclosure also provides a method for preparing the compound hydrocortisone and neomycin sulfate granule according to the above technical solutions, including the following steps:
(1) adding the acetaminophen and the inclusion stabilizer to water to form a solution 1;
(2) adding the chlorpheniramine maleate and the methylephedrine hydrochloride to water to form a solution 2; and
(3) mixing the solution 1 and the solution 2, adding the artificial calculus bovis and the caramel, and mixing to be uniform to obtain a soft material; and subjecting the soft material to granulating, drying, and screening in sequence to obtain the compound hydrocortisone and neomycin sulfate granule.

The present disclosure also provides a use of the compound hydrocortisone and neomycin sulfate granule according to the above technical solutions in preparation of a drug for preventing and/or treating a cold.

In some embodiments, the cold is selected from the group consisting of a common cold and an influenza.

The present disclosure also provides use of the compound hydrocortisone and neomycin sulfate granule according to the above technical solutions in prevention and/or treatment of a cold.

Embodiments of the present disclosure have the following technical effects:

Acetaminophen and chlorpheniramine maleate have poor photostability and thus exhibit unsatisfactory quality stability. In the present disclosure, natural Chinese medicinal herbs are subjected to extraction to obtain natural stabilizers that could stabilize low-concentration acetaminophen and chlorpheniramine maleate. The natural stabilizers extracted from the natural Chinese medicinal herbs are encapsulated with hydroxypropyl-ß-cyclodextrin with specific DS, which could achieve the effective light shielding of acetaminophen and chlorpheniramine maleate with poor photostability to ensure the prominent stability of acetaminophen and chlorpheniramine maleate at low concentrations. Furthermore, the effective stabilization of bilirubin as an active ingredient in the artificial calculus bovis according to the present disclosure could reduce a loss rate of the bilirubin. Moreover, the addition of the caramel further ensures a stable quality system of the components.

The present disclosure guarantees the stability of low-concentration chlorpheniramine maleate and ephedrine in a preparation, thereby avoiding the side effects and adverse reactions caused by high-concentration chlorpheniramine maleate and ephedrine. Therefore, the present disclosure has great industrial application and promotion values.

### DETAILED DESCRIPTION OF THE EMBODIMENTS

Several exemplary embodiments of the present disclosure are now described in detail. The detailed description should not be considered as a limitation to the present disclosure, but should be understood as a more detailed description of some aspects, features, and implement solutions of the present disclosure.

It should be understood that terms described in the present disclosure are merely used to describe specific embodiments and are not intended to limit the present disclosure. In addition, for a numerical range in the present disclosure, it should be understood that each intermediate value between an upper limit and a lower limit of the range is also specifically disclosed. Each small range between any stated value or an intermediate value in a stated range and any other stated value or an intermediate value in the stated range is also included in the present disclosure. Upper and lower limits of each of these small ranges can independently be included in or excluded from the range.

Unless otherwise stated, all technical and scientific terms used herein have the same meaning as commonly understood by those of ordinary skill in the art described in the present disclosure. Although only preferred methods and materials are described in the present disclosure, any methods and materials similar or equivalent to those described herein could also be used in the practice or testing of the present disclosure. All documents mentioned in this specification are incorporated by reference to disclose and describe methods and/or materials related to the documents. In case of conflict with any incorporated documents, the content of this specification shall prevail.

It is obvious to a person skilled in the art that a plurality of modifications and variations could be made to the specific embodiments of the present specification without departing from the scope or spirit of the present disclosure. Other embodiments derived from the description of the present disclosure are obvious to a person skilled in the art. The specification and embodiments of the present disclosure are merely exemplary.

As used herein, the terms such as “including”, “having”, and “containing” are all open-ended terms, which means including but not limited to.

### Example 1

(1) A white peony root was crushed to 200 mesh and then mixed with water according to a mass-volume ratio of 1g: 50 mL to obtain a mixed system. Then the mixed system was filled in a flexible packaging material and sealed. A pressure of 120 MPa was applied continuously for 1 min, and then a pressure of 100 MPa was applied continuously for 0.5 min. A pressure relief was conducted, a resulting system was subjected to filtration to obtain a filtrate, and the filtrate was collected.

(2) The filtrate was concentrated to a volume 50% of the original volume, and ethanol was added until an alcohol content was 42%. The resulting system was subjected to standing and centrifugation to obtain a precipitate, and the precipitate was discarded to obtain a supernatant. Ethanol was further added to the supernatant until an alcohol content was 55%. An obtained system was subjected to standing and vacuum filtration to obtain a filter cake. The filter cake was collected and dried to obtain a white peony root extract.

### Example 2

Lcorice was crushed and added to a reactor that was preheated to a temperature of 50° C. to 60° C., and water was added as an extraction solvent to the reactor. A pressure of the reactor was raised to 30 MPa, carbon dioxide was introduced into the reactor continuously for 30 min. A pressure relief was conducted to obtain a reaction system. The reaction system was concentrated and dried to obtain a licorice extract.

A volume ratio of a mixture of the licorice and the water to the carbon dioxide was 1:3.

### Example 3 Preparation of an Inclusion Stabilizer

A white peony root extract and a licorice extract were encapsulated with hydroxypropyl-β-cyclodextrin (DS=4.2):

A mass ratio of the white peony root extract to the licorice extract was 1:1.5.

(1) The hydroxypropyl-β-cyclodextrin was dissolved in phosphoric acid with a pH of 2.0 to prepare a solution of 4.8 wt %.

(2) The white peony root extract and the licorice extract were dissolved with anhydrous ethanol to obtain a mixed extract solution. The mixed extract solution was slowly added dropwise to the solution obtained in step (1). A resulting mixture was subjected to stirring in a nitrogen atmosphere to and inclusion for 7.5 h to obtain a reaction system.

(3) The reaction system obtained in step (2) was centrifuged to obtain a supernatant. The supernatant was collected and lyophilized to obtain the inclusion stabilizer.

### Example 4

A compound hydrocortisone and neomycin sulfate granule consist of the following components by mass:
140 g of acetaminophen, 1 g of chlorpheniramine maleate, 1.2 g of methylephedrine hydrochloride, 2 g of an inclusion stabilizer, 3.0 g of artificial calculus bovis, and 0.5 g of a caramel.

The inclusion stabilizer was the inclusion stabilizer prepared in Example 3.

A method for preparing the compound hydrocortisone and neomycin sulfate granule was conducted by the following steps:
(1) The acetaminophen and the inclusion stabilizer were added to 250 ml of water to form a solution 1.
(2) The chlorpheniramine maleate and the methylephedrine hydrochloride were added to 1,500 mL of water to form a solution 2.
(3) The solution 1 and the solution 2 were mixed. The artificial calculus bovis and the caramel were added thereto, and mixed to be uniform to obtain a soft material. The soft material was granulated, dried, and screened to obtain the compound hydrocortisone and neomycin sulfate granule.

### Example 5

A compound hydrocortisone and neomycin sulfate granule, including the following components by mass:

140 g of acetaminophen, 1 g of chlorpheniramine maleate, 1.2 g of methylephedrine hydrochloride, 2.5 g of an inclusion stabilizer, 3.0 g of artificial calculus bovis, and 1 g of a caramel.

The inclusion stabilizer was the inclusion stabilizer prepared in Example 3.

A method for preparing the compound hydrocortisone and neomycin sulfate granule was conducted by the following steps:
(1) The acetaminophen and the inclusion stabilizer were added to 250 mL of water to form a solution 1.
(2) The chlorpheniramine maleate and the methylephedrine hydrochloride were added to 1,500 mL of water to form a solution 2.
(3) The solution 1 and the solution 2 were mixed. The artificial calculus bovis and the caramel were added thereto, and mixed to be uniform to obtain a soft material. The soft material was granulated, dried, and screened to obtain the compound hydrocortisone and neomycin sulfate granule.

### Example 6

A compound hydrocortisone and neomycin sulfate granule, including the following components by mass:

140 g of acetaminophen, 1 g of chlorpheniramine maleate, 1.2 g of methylephedrine hydrochloride, 2.5 g of an inclusion stabilizer, 3.0 g of artificial calculus bovis, and 0.6 g of a caramel.

The inclusion stabilizer was the inclusion stabilizer prepared in Example 3.

A method for preparing the compound hydrocortisone and neomycin sulfate granule was conducted by the following steps:
(1) The acetaminophen and the inclusion stabilizer were added to 250 mL of water to form a solution 1.
(2) The chlorpheniramine maleate and the methylephedrine hydrochloride were added to 1,500 mL of water to form a solution 2.
(3) The solution 1 and the solution 2 were mixed. The artificial calculus bovis and the caramel were added thereto, and mixed to be uniformmixing to obtain a soft material. The soft material was granulated, dried, and screened to obtain the compound hydrocortisone and neomycin sulfate granule.

### Example 7

A compound hydrocortisone and neomycin sulfate granule, including the following components by mass:

140 g of acetaminophen, 1 g of chlorpheniramine maleate, 1.2 g of methylephedrine hydrochloride, 2.4 g of an inclusion stabilizer, 3.0 g of artificial calculus bovis, and 0.7 g of a caramel.

The inclusion stabilizer was the inclusion stabilizer prepared in Example 3.

A method for preparing the compound hydrocortisone and neomycin sulfate granule was conducted by the following steps:
(1) The acetaminophen and the inclusion stabilizer were added to 250 ml of water to form a solution 1.
(2) The chlorpheniramine maleate and the methylephedrine hydrochloride were added to 1,500 mL of water to form a solution 2.
(3) The solution 1 and the solution 2 were mixed. The artificial calculus bovis and the caramel were added thereto, and mixed to be uniform to obtain a soft material. The soft material was granulated, dried, and screened to obtain the compound hydrocortisone and neomycin sulfate granule.

### Example 8

A compound hydrocortisone and neomycin sulfate granule, including the following components by mass:
140 g of acetaminophen, 1 g of chlorpheniramine maleate, 1.2 g of methylephedrine hydrochloride, 2.3 g of an inclusion stabilizer, 3.0 g of artificial calculus bovis, and 0.8 g of a caramel.

The inclusion stabilizer was the inclusion stabilizer prepared in Example 3.

A method for preparing the compound hydrocortisone and neomycin sulfate granule was conducted by the following steps:
(1) The acetaminophen and the inclusion stabilizer were added to 250 mL of water to form a solution 1.
(2) The chlorpheniramine maleate and the methylephedrine hydrochloride were added to 1,500 mL of water to form a solution 2.
(3) The solution 1 and the solution 2 were mixed. The artificial calculus bovis and the caramel were added thereto, and mixed to be uniform to obtain a soft material. The soft material was granulated, dried, and screened to obtain the compound hydrocortisone and neomycin sulfate granule.

### Comparative Example 1

This comparative example was conducted as described in Example 4 except that the natural stabilizers encapsulated in the inclusion stabilizer were all licorice extracts (a total mass of the natural stabilizers remained unchanged).

### Comparative Example 2

This comparative example was conducted as described in Example 4 except that DS of the hydroxypropyl-β-cyclodextrin was 5.4.

### Comparative Example 3

This comparative example was conducted as described in Example 4 except that the caramel was not added.

### Effect Verification Example 1

The inclusion stabilizer prepared in Example 3 was accurately weighed. Anhydrous ethanol was added. And a resulting mixture was subjected to ultrasonic treatment for 30 min to obtain a dispersion. Then the dispersion was transferred to a brown volumetric flask, and anhydrous ethanol was added thereto to a specified scale mark. Using anhydrous ethanol as a blank control, the absorbance of an inclusion compound was measured at a wavelength of 423 nm. A content of natural stabilizers in the inclusion compound was calculated according to a regression equation, so as to obtain an inclusion rate of the inclusion stabilizer. Results show that inclusion rates of Examples 3 to 7 of the present disclosure are all 98% or more.

### Effect Verification Example 2

Experimental scheme: 50 healthy and mature Institute of Cancer Research (ICR) mice each with a body weight of 20 g to 25 g were selected, consisting of 25 male mice and 25 female mice. The mice were fasted without water deprivation for 12 h. The mice were intragastrically administered at 20 g/kg BW 3 times within 1 day, that is, the intragastric administration was conducted once every 8 h. After intragastric administration for 2 h, the mice were allowed to eat and drink freely. The poisoning and death manifestations of the mice were recorded consecutively for 7 d.

Experimental drug: The compound hydrocortisone and neomycin sulfate granule prepared in Example 4.

Experimental results are shown in Table 1 (during the experiment, no poisoning occurred in the mice).

TABLE 1
Poisoning and death manifestations of mice administered with the compound
hydrocortisone and neomycin sulfate granule prepared in Example 4

| Gender | Initial body weight (g) | Final body weight (g) | Death (mice) |
|---|---|---|---|
| Female | 21.2 ± 0.2 | 20.8 ± 0.3 | 0 |
| Male | 23.4 ± 0.3 | 24.1 ± 0.6 | 0 |

### Effect Verification Example 3

According to the sampling method specified in the Chinese Pharmacopoeia, 5 samples were collected from each of the compound hydrocortisone and neomycin sulfate granules in Examples 4 to 8. And colors of the samples were observed. Results show that each sample is slightly yellowish. The results indicate that characteristics of the compound hydrocortisone and neomycin sulfate granule prepared according to the present disclosure all meet the requirements of national standards.

### Effect Verification Example 4

According to the sampling method specified in the Chinese Pharmacopoeia, 5 samples were collected from each of the compound hydrocortisone and neomycin sulfate granules in Examples 4 to 8 of the present disclosure. 10 g was collected from each sample and the solubility of the samples were tested.

The testing was conducted by the solubility test method under the granule item in the Chinese Pharmacopoeia (Part IV, 2015 edition). Results show that the compound hydrocortisone and neomycin sulfate granules in Examples 4 to 8 of the present disclosure are all qualified.

### Effect Verification Example 5

A content of chlorpheniramine maleate in a compound hydrocortisone and neomycin sulfate granule was detected by high-performance liquid chromatography. A mass proportion (%) of the chlorpheniramine maleate in the compound hydrocortisone and neomycin sulfate granule relative to the raw material chlorpheniramine maleate was calculated. 5 samples were collected for each test group. Test results are shown in Table 2.

TABLE 2
Contents of chlorpheniramine maleate in the compound
hydrocortisone and neomycin sulfate granules prepared in
the examples and comparative examples

| | Sample 1 | Sample 2 | Sample 3 | Sample 4 | Sample 5 |
|---|---|---|---|---|---|
| Example 4 | 99.82 | 99.76 | 99.86 | 99.84 | 99.79 |
| Example 5 | 99.81 | 99.67 | 99.79 | 99.77 | 99.78 |
| Example 6 | 99.68 | 99.65 | 99.67 | 99.85 | 99.76 |
| Example 7 | 99.80 | 99.84 | 99.64 | 99.77 | 99.85 |
| Example 8 | 99.66 | 99.78 | 99.82 | 99.74 | 99.71 |
| Comparative | 91.36 | 91.61 | 92.17 | 92.32 | 91.84 |
| Example 1 | | | | | |
| Comparative | 92.64 | 92.36 | 91.39 | 91.54 | 91.47 |
| Example 2 | | | | | |
| Comparative | 95.31 | 96.24 | 95.26 | 94.38 | 94.91 |
| Example 3 | | | | | |

It can be seen that the present disclosure can achieve the high stability of chlorpheniramine maleate at an ultra-low content, thereby ensuring the quality stability and high efficiency of a compound hydrocortisone and neomycin sulfate granule.

### Effect Verification Example 6

A content of methylephedrine hydrochloride in a compound hydrocortisone and neomycin sulfate granule was detected by high-performance liquid chromatography. A mass ratio of the methylephedrine hydrochloride in the compound hydrocortisone and neomycin sulfate granule relative to the raw material methylephedrine hydrochloride was calculated. 5 samples were collected for each test group. Test results are shown in Table 3.

TABLE 3
Contents of methylephedrine hydrochloride in the compound
hydrocortisone and neomycin sulfate granules prepared in the
examples and comparative examples

| | Sample 1 | Sample 2 | Sample 3 | Sample 4 | Sample 5 |
|---|---|---|---|---|---|
| Example 4 | 99.86 | 99.82 | 99.84 | 99.75 | 99.74 |
| Example 5 | 99.78 | 99.79 | 99.52 | 99.76 | 99.67 |
| Example 6 | 99.84 | 99.78 | 99.67 | 99.65 | 99.58 |
| Example 7 | 99.76 | 99.56 | 99.45 | 99.81 | 99.61 |
| Example 8 | 98.81 | 99.82 | 99.45 | 99.45 | 99.80 |
| Comparative | 90.25 | 91.34 | 92.32 | 90.52 | 91.56 |
| Example 1 | | | | | |
| Comparative | 91.36 | 90.38 | 92.58 | 91.24 | 91.84 |
| Example 2 | | | | | |
| Comparative | 93.25 | 93.68 | 94.59 | 93.81 | 92.79 |
| Example 3 | | | | | |

It can be seen that the present disclosure can maintain the efficacy stability of methylephedrine hydrochloride at an ultra-low content, thereby ensuring the quality stability and high efficiency of a compound hydrocortisone and neomycin sulfate granule.

Effect Verification Example 7

A content of bilirubin in a preparation was determined by high-performance liquid chromatography. A loss rate of bilirubin in a finished product was calculated based on an amount of bilirubin in the artificial calculus bovis raw material to prepare a compound hydrocortisone and neomycin sulfate granule. Results are as follows:

TABLE 4
Loss rates of bilirubin in the compound hydrocortisone and neomycin sulfate
granules prepared in the examples and the comparative examples

| | | | | | | Com- | Com- | Com- |
|---|---|---|---|---|---|---|---|---|
| | | | | | | parative | parative | parative |
| | Example 4 | Example 5 | Example 6 | Example 7 | Example 8 | Example 1 | Example 2 | Example 3 |
| Bilirubin | 1.10 | 1.02 | 1.04 | 1.05 | 1.02 | 10.25 | 9.58 | 7.69 |
| loss | | | | | | | | |
| rate (%) | | | | | | | | |

The above are merely preferred embodiments of the present disclosure rather than limitations to the present disclosure in any form. It should be noted that those of ordinary skill in the art may further make several improvements and modifications without departing from the principle of the present disclosure, but such improvements and modifications should be deemed as falling within the scope of the present disclosure.

1. A compound hydrocortisone and neomycin sulfate granule, comprising the following components in parts by mass:
140 parts of acetaminophen, 1 part of chlorpheniramine maleate, 1.2 parts of methylephedrine hydrochloride, 2 to 2.5 parts of an inclusion stabilizer, 3.0 parts of artificial calculus bovis, and 0.5 to 1 parts of a caramel,
wherein the inclusion stabilizer is produced by encapsulating a white peony root extract and a licorice extract with hydroxypropyl-β-cyclodextrin.

2. The compound hydrocortisone and neomycin sulfate granule of claim 1, comprising the following components in parts by mass:
140 parts of the acetaminophen, 1 part of the chlorpheniramine maleate, 1.2 parts of the methylephedrine hydrochloride, 2 parts of the inclusion stabilizer, 3.0 parts of the artificial calculus bovis, and 0.5 parts of the caramel.

3. The compound hydrocortisone and neomycin sulfate granule of claim 1, comprising the following components in parts by mass:
140 parts of the acetaminophen, 1 part of the chlorpheniramine maleate, 1.2 parts of the methylephedrine hydrochloride, 2.5 parts of the inclusion stabilizer, 3.0 parts of the artificial calculus bovis, and 1 part of the caramel.

4. The compound hydrocortisone and neomycin sulfate granule of claim 1, comprising the following components in parts by mass:
140 parts of the acetaminophen, 1 part of the chlorpheniramine maleate, 1.2 parts of the methylephedrine hydrochloride, 2.5 parts of the inclusion stabilizer, 3.0 parts of the artificial calculus bovis, and 0.6 parts of the caramel.

5. The compound hydrocortisone and neomycin sulfate granule of claim 1, comprising the following components in parts by mass:
140 parts of the acetaminophen, 1 part of the chlorpheniramine maleate, 1.2 parts of the methylephedrine hydrochloride, 2.5 parts of the inclusion stabilizer, 3.0 parts of the artificial calculus bovis, and 0.7 parts of the caramel.

6. The compound hydrocortisone and neomycin sulfate granule of claim 1, comprising the following components in parts by mass:
140 parts of the acetaminophen, 1 part of the chlorpheniramine maleate, 1.2 parts of the methylephedrine hydrochloride, 2.5 parts of the inclusion stabilizer, 3.0 parts of the artificial calculus bovis, and 0.8 parts of the caramel.

7. The compound hydrocortisone and neomycin sulfate granule of claim 1, wherein the white peony root extract is extracted by a process comprising the following steps:
(1) crushing a white peony root to obtain a crushed material, and mixing the crushed material with water to obtain a mixed system; applying a pressure of 120 MPa to 125 MPa continuously for 1 min to 1.5 min and then a pressure of 100 MPa to 105 MPa continuously for 0.5 min to 1 min to the mixed system; and subjecting a resulting system to pressure relief, and then filtration to obtain a filtrate, and collecting the filtrate; and
(2) adjusting an alcohol content of the filtrate to 40% to 42%, standing to produce a precipitate, and discarding the precipitate to obtain a supernatant; further adjusting an alcohol content of the supernatant to 55% to 57%, and subjecting an obtained system to standing and then vacuum filtration to obtain a filter cake; and collecting and drying the filter cake to obtain the white peony root extract.

8. The compound hydrocortisone and neomycin sulfate granule of claim 7, wherein a ratio of a mass of the white peony root to a volume of the water is 1 g: 50 mL.

9. The compound hydrocortisone and neomycin sulfate granule of claim 1, wherein the licorice extract is extracted by a process comprising the following steps:
crushing licorice, and adding water as an extraction solvent to a resulting crushed licorice to obtain a mixture; introducing carbon dioxide into the mixture at a temperature of 50° C. to 60° C. and a pressure of 10 MPa to 40 MPa for 20 min to 40 min, and conducting pressure relief to obtain a reaction system; and concentrating and drying the reaction system to obtain the licorice extract.

10. The compound hydrocortisone and neomycin sulfate granule of claim 9, wherein a volume ratio of a mixture of the licorice and the water to the carbon dioxide is in a range of 1:1 to 1:5.

11. The compound hydrocortisone and neomycin sulfate granule of claim 1, wherein the inclusion stabilizer is prepared by a process comprising the following steps:
(1) dissolving the hydroxypropyl-β-cyclodextrin in an acid solution with a pH of 2.0 to form a solution with a concentration of 4.5 wt % to 5.0 wt %;
(2) dissolving the white peony root extract and the licorice extract in anhydrous ethanol to obtain a mixed extract solution, adding the mixed extract solution dropwise to the solution obtained in step (1), and subjecting a resulting mixture to stirring in a nitrogen atmosphere and inclusion for 7 h to 8 h to obtain a reaction system; and
(3) centrifuging the reaction system obtained in step (2) to obtain a supernatant, and collecting and lyophilizing the supernatant to obtain the inclusion stabilizer.

12. The compound hydrocortisone and neomycin sulfate granule of claim 1, wherein a mass ratio of the white peony root extract to the licorice extract is 1:1.5.

13. The compound hydrocortisone and neomycin sulfate granule of claim 1, wherein a degree of substitution (DS) of the hydroxypropyl-β-cyclodextrin is 4.2.

14. A method for preparing the compound hydrocortisone and neomycin sulfate granule of claim 1, comprising the following steps:
(1) adding the acetaminophen and the inclusion stabilizer to water to form a solution 1;
(2) adding the chlorpheniramine maleate and the methylephedrine hydrochloride to water to form a solution 2; and
(3) mixing the solution 1 and the solution 2, adding the artificial calculus bovis and the caramel and mixing to be uniform to obtain a soft material; and subjecting the soft material to granulating, drying, and screening in sequence to obtain the compound hydrocortisone and neomycin sulfate granule.

15. A method for preventing and/or treating a cold, comprising administrating a therapeutic effective amount of the compound hydrocortisone and neomycin sulfate granule of claim 1 to a subject in need thereof.

16. The method of claim 15, wherein the cold is selected from the group consisting of a common cold and an influenza.

17. (canceled)

18. The compound hydrocortisone and neomycin sulfate granule of claim 2, wherein the white peony root extract is extracted by a process comprising the following steps:
(1) crushing a white peony root to obtain a crushed material, and mixing the crushed material with water to obtain a mixed system; applying a pressure of 120 MPa to 125 MPa continuously for 1 min to 1.5 min and then a pressure of 100 MPa to 105 MPa continuously for 0.5 min to 1 min to the mixed system; and subjecting a resulting system to pressure relief, and then filtration to obtain a filtrate, and collecting the filtrate; and
(2) adjusting an alcohol content of the filtrate to 40% to 42%, standing to produce a precipitate, and discarding the precipitate to obtain a supernatant; further adjusting an alcohol content of the supernatant to 55% to 57%, and subjecting an obtained system to standing and then vacuum filtration to obtain a filter cake; and collecting and drying the filter cake to obtain the white peony root extract.

19. The compound hydrocortisone and neomycin sulfate granule of claim 3, wherein the white peony root extract is extracted by a process comprising the following steps:
(1) crushing a white peony root to obtain a crushed material, and mixing the crushed material with water to obtain a mixed system; applying a pressure of 120 MPa to 125 MPa continuously for 1 min to 1.5 min and then a pressure of 100 MPa to 105 MPa continuously for 0.5 min to 1 min to the mixed system; and subjecting a resulting system to pressure relief, and then filtration to obtain a filtrate, and collecting the filtrate; and
(2) adjusting an alcohol content of the filtrate to 40% to 42%, standing to produce a precipitate, and discarding the precipitate to obtain a supernatant; further adjusting an alcohol content of the supernatant to 55% to 57%, and subjecting an obtained system to standing and then vacuum filtration to obtain a filter cake; and collecting and drying the filter cake to obtain the white peony root extract.

20. The compound hydrocortisone and neomycin sulfate granule of claim 11, wherein a mass ratio of the white peony root extract to the licorice extract is 1:1.5.

21. The compound hydrocortisone and neomycin sulfate granule of claim 11, wherein a degree of substitution (DS) of the hydroxypropyl-β-cyclodextrin is 4.2.
